(19) United States
(12) Patent Application Publication
(10) Pub. No.: US 2019/0240654 A1
(43) Pub. Date: Aug. 8, 2019

(54) METHOD AND APPARATUS FOR MUTLI-CHANNEL URINALYSIS TEST KIT WITH MULTI-FACTOR CONTROL

(71) Applicant: Spectrum Laboratories, LLC, Cincinnati, OH (US)

(72) Inventor: James Matthew Stephens, Cincinnati, OH (US)

(21) Appl. No.: 16/269,024

(22) Filed: Feb. 6, 2019

Related U.S. Application Data
(60) Provisional application No. 62/627,447, filed on Feb. 7, 2018.

(51) Int. Cl. B01L 3/00; A61B 10/00

(52) U.S. Cl. CPC B01L 3/502; A61B 10/007; A61B 2010/0009; B01L 2400/0406; B01L 2300/0663

(57) ABSTRACT

A system and method for increasing confidence, privacy, and efficiency in obtaining in-home urinalysis results is contemplated. A negative-result control is provided in conjunction with a plurality of test cartridges designed to yield results about the test sample in several different regards (e.g., use of selected combinations of drugs and/or alcohol by the test subject, the integrity of the urine sample, the medical or physiological condition(s) of the test subject, etc.). Convenient sample test bins are included to allow for real-time and/or side-by-side comparison of the urine sample test results against a verified, negative result control.

### CROSS REFERENCE TO RELATED APPLICATIONS

This application claims priority to U.S. provisional patent application Ser. No. 62/627,447 filed on Feb. 7, 2018 under the same title and inventor. The text of this application is incorporated by reference herein.

### TECHNICAL FIELD

The present invention relates generally to improved methods and apparatus for quick, convenient, and verifiable urinalysis and, more particularly, to a kit having a multi-channel tester used in combination with a multi-factor negative control to deliver near-instantaneous, verified results. The test kit may be tailored to an individual's circumstances, and the multi-channel nature allows for the design of in-home, verifiable testing to provide a more comprehensive understanding of a subset of related conditions (and/or to concurrently identify related risk-factors).

### BACKGROUND

Urinalysis has become the preferred method for testing humans with respect to their previous ingestion of any number of substances. In practice, urinalysis involves testing for specific chemical components, also referred to as analytes, that are the residuals from digestion and/or ingestion of those compounds. In turn, the compounds are usually specific to a certain food, drug or other substance. Thus, urinalysis provides an inferential means of detecting prior use or ingestion of the substance, but without directly testing for that substance itself.

Urinalysis has found particular utility in identifying persons who use nicotine, medications, performance enhancing drugs, opioids, and other controlled substances. Individuals are required to urinate into a clean, uncontaminated specimen collector, such as a plastic cup or beaker. A technician then seals the specimen and transports it to an analytical lab, where any number of instruments may be employed to detect the presence of analytes of interest. When performed in a professional laboratory setting, it is common practice for technicians to verify positive tests against a known source of the analyte, thereby providing a comparative confirmation of the positive result.

This system has a number of drawbacks. Foremost, the specimen is at risk of becoming lost or contaminated, owing to the passage of time and the necessity to properly seal, label, track, and transport the specimen. Further, the reliance on laboratory services and technicians removes a sense of privacy that may be important to some individuals, such as recovering addicts or others who simply prefer to avoid having a record of the various analytes detected. To the extent positive results are tested against known analytes (i.e., a positive control sample), the fact some analytes relate to controlled substances (e.g., some form of narcotics, opioids, etc.) means that the analyses may be similarly restricted or controlled, thereby requiring testing to be carried out at a small number of designated facilities. Finally, these services inherently involve delays to the extent the specimens must be transported, analyzed, and then reported back to the individual (and/or employers, guardians, and other service providers).

Another flaw to this approach is that some analytes are not produced exclusively by a single substance (i.e., the body may create the same or confusingly similar analytes upon ingestion of multiple, different substances). Further, given the time and additional handling procedures required to transfer and test a sample, confirmation of a positive test by way of retesting takes even more time. However, natural metabolic processes cause most analytes to be eliminated from the body over time, so this type of iterative retesting regimens creates uncertainty in urinalysis, both in terms of identifying false positive results (e.g., owing to contamination of the sample) and verifying accurate results.

Accordingly, a number of alternative arrangements for urinalysis have been proposed. Some of these alternative systems rely on simplified detectors for a specific analyte and/or improved means for collecting and submitting the specimens. To the best of the inventor's knowledge, all of these solutions rely on a single, unconfirmed analysis or, at least implicitly, an iterative, “test-and-retest” regimen when it comes to dealing with a potential positive result.

Individual test strips and home use cartridges are available. These systems include a one or a series of chemically treated test papers positioned within a tester. The user must provide a sample and then dip the one end of the strip/cartridge into the sample, thereby running a risk for contamination to the extent a clean, sterilized sample cup is not used. Worse still, in the event the collection process is flawed and/or the sample itself becomes contaminated, the user has no way of immediately verifying this fact, especially to the extent providing positive result verification may be impractical or, with respect to certain substances, illegal.

U.S. Pat. No. 5,976,895 describes a drug abuse test kit with a cup-like container for retaining a fluid sample to be tested, as seen in FIG. 1. A test strip card 25 is inserted through a slit form on the inner closure insert 15. Card 25 may comprise a multiple drug test card having various test strips with visual endpoints to indicate the presence or absence of a particular drug. A cap 23 having a textured surface 22 with sidewalls 24 screws onto corresponding screws 21 in the insert 15 and/or sidewalls 11, 12 of the cup 10. Bottom 13 helps to hermetically seal the cup 10 when the cap 23 is affixed.

United State Patent Publication 2004/0176705 envisions a cartridge-style, integrated fluid collection and testing system. A disposable device includes an array of electrical contacts, and electrochemical sensors that may be coupled to an analytical device, such as a computer, to control testing of the samples and to provide for a rapid indication of test results at the point of care.

U.S. Pat. No. 4,473,530 discloses a closed integrated system for handling urine samples to minimize the risk of contamination. A clear, tubular, disposable container is subdivided into a larger main longitudinal chamber integrated with adjacent chambers therein. Reagent strips and/or a hydrometer may be contained within the chambers.

### SUMMARY OF INVENTION

Specific reference is made to the appended information, including any and all claims, drawings, and description, which disclose elements of the invention. While specific embodiments are identified, it will be understood that elements from one described aspect may be combined with those from a separately identified aspect. In the same manner, a person of ordinary skill will have the requisite understanding of common processes, components, and methods, and this description is intended to encompass and disclose such common aspects even if they are not expressly identified herein.

In one embodiment, the invention is a urinalysis home-testing kit having any combination of the following features:
a cartridge holder holding a plurality of urinalysis test strips in a spaced apart array, the holder having a detachable end cap fitting over the test strips;
a multi-factor negative-result control sample;
wherein the multi-factors are selected to mimic human urine;
a sterilized specimen collection cup;
a sterilized control comparison cup;
wherein the specimen collection cup and the control comparison cup are substantially identical;
wherein there are at least ten test strips and the array is a 5×2 matrix;
wherein the test strips indicate analytes associated with any combination of the following substances: nicotine, tetrahydrocannabinol, cocaine, amphetamine, methamphetamine, opioid analgesics, phencyclidine, methylenedioxymethamphetamine (MDMA), barbiturate, and benzodiazepines;
wherein the cartridge holder is contained in a hermetically sealed pouch, including a moisture absorbent;
wherein the collection cup and/or the control comparison cup has a sufficient diameter and depth to receive the plurality of urinalysis test strips without having any single test strip come into contact with a sidewall of the cup or cups;
wherein the cartridge holder includes an aperture to allow a user to view results indicia produced by a single test strip associated with the aperture;
wherein a plurality of apertures are associated with a plurality of single test strips;
wherein the multi-factor negative-result control sample includes shelf-life extenders and said shelf-life extenders do not interfere with production of accurate results for the test kit/urinalysis; and
packaging to fully contain any combination of the multi-factor negative-result control sample, the cartridge holder, the collection cup, the control comparison, and instructions for using the kit.

In another embodiment, the invention is a method for conducing urinalysis to produce instant results having any combination of the following features:
providing a negative-result control and a plurality of multi-channel urinalysis devices within one or more hermetically sealed packages;
instructing a user to provide a urine sample into a sterilized container and subsequently removing the urinalysis device from the package and contacting the urinalysis device with the urine sample;
in the event of a positive test indication by the urinalysis device, instructing the user to repeat the removing and contacting steps with a second urinalysis device and the control sample;
wherein the control sample includes a multi-factor combination of substances selected to mimic human urine; and
wherein the multi-factor combination also includes a shelf-life extender.

### DESCRIPTION OF THE DRAWINGS

Operation of the invention may be better understood by reference to the detailed description taken in connection with the following illustrations. These appended drawings form part of this specification, and any information on/in the drawings is both literally encompassed (i.e., the actual stated values) and relatively encompassed (e.g., ratios for respective dimensions of parts). In the same manner, the relative positioning and relationship of the components as shown in these drawings, as well as their function, shape, dimensions, and appearance, may all further inform certain aspects of the invention as if fully rewritten herein. Unless otherwise stated, all dimensions in the drawings are with reference to inches, and any printed information on/in the drawings form part of this written disclosure.

In the drawings and attachments, all of which are incorporated as part of this disclosure:

FIG. 1 is a perspective illustration of a urinalysis apparatus according to the prior art.

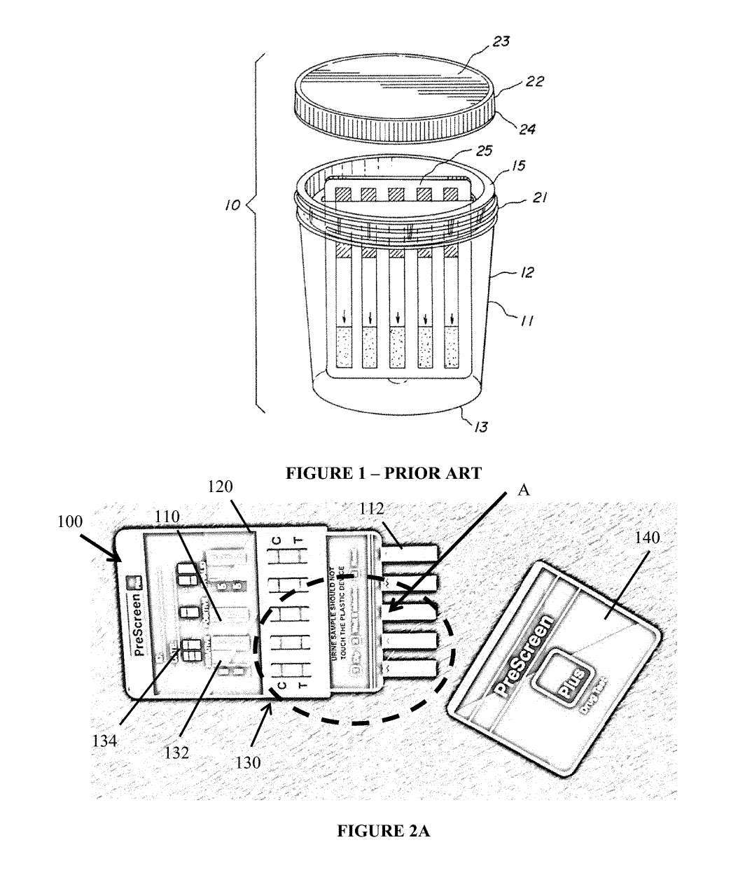

FIG. 2A is an illustration of a multi-channel tester component in its detached or open state, as contemplated in certain embodiments. FIG. 2B is a cross sectional view of call-out A from FIG. 2A. FIG. 2C is an illustration of the tester of FIG. 2A but in its attached or closed state.

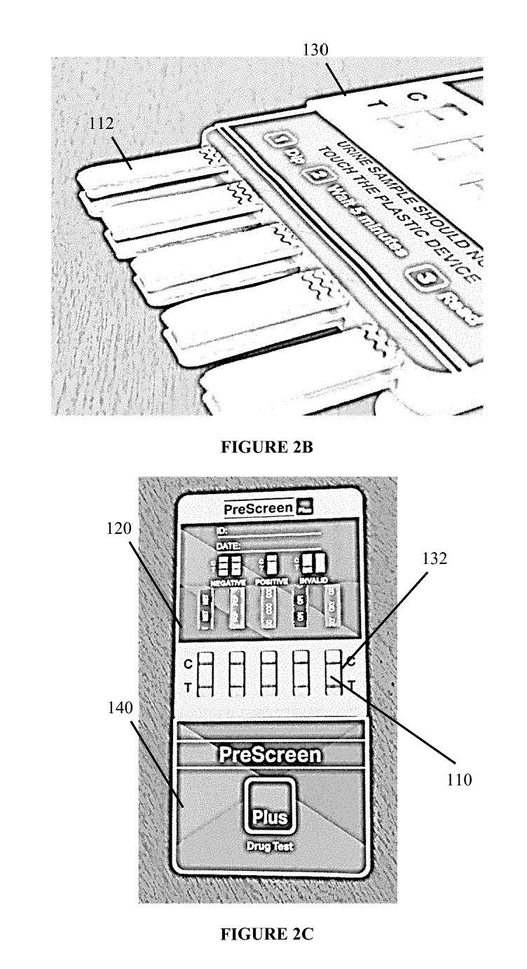

FIGS. 3A through 3B are illustrations of various test bin components, as contemplated in certain embodiments.

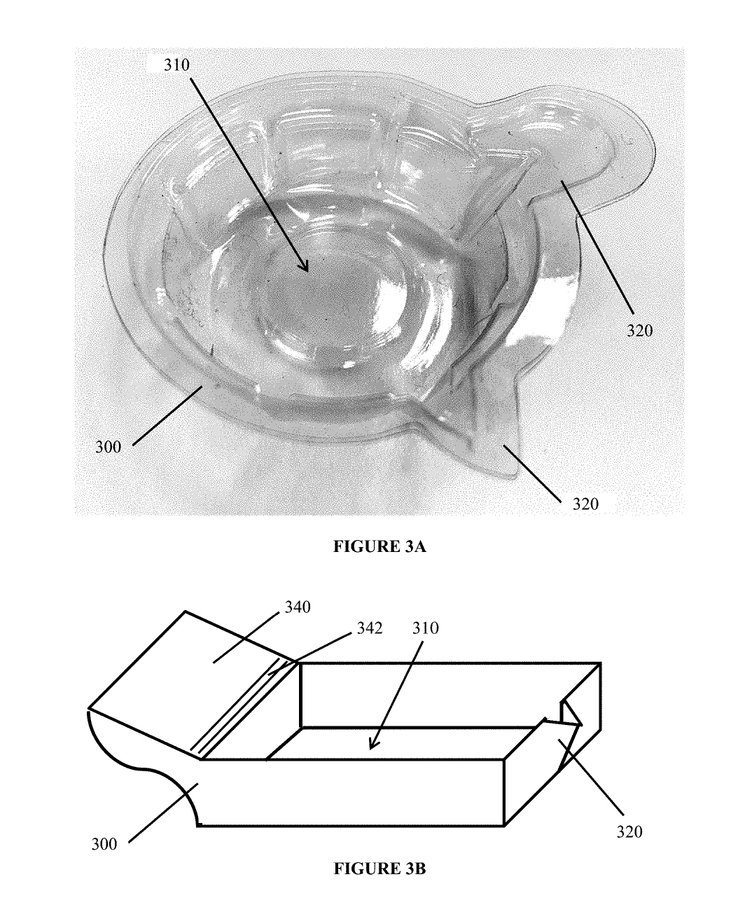

FIG. 4 is an illustration of the testing kit, as contemplated in certain embodiments, including a negative control container, a test bin, a foil-wrapped multi-channel tester, a desiccant, and a storage box.

### DETAILED DESCRIPTION

Reference will now be made in detail to exemplary embodiments of the present invention, examples of which are illustrated in the accompanying drawings. It is to be understood that other embodiments may be utilized and structural and functional changes may be made without departing from the respective scope of the invention. As such, the following description is presented by way of illustration only and should not limit in any way the various alternatives and modifications that may be made to the illustrated embodiments and still be within the spirit and scope of the invention.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

The invention described herein comprises a home-testing kit specifically designed for an unskilled user (i.e., a person lacking formal training or familiarity with urinalysis procedures). The kit includes a number of fail-safes to ensure, in the event of a positive result, the user can quickly and accurately verify whether the positive result was based upon the sample or, instead, based upon some user error or contaminated/expired testing equipment.

Owing to the complex chemical nature of urine, the use of simple water does not provide a comparable negative control. Further, as noted above, positive control is not always feasible or desirable. Thus, the inventive system and method relies on a multi-channel urinalysis kit that includes a verified negative control sample to address these issues. Further, it can be used in the privacy of the user's home, without engaging outside laboratory services and the delays in time inherent thereto.

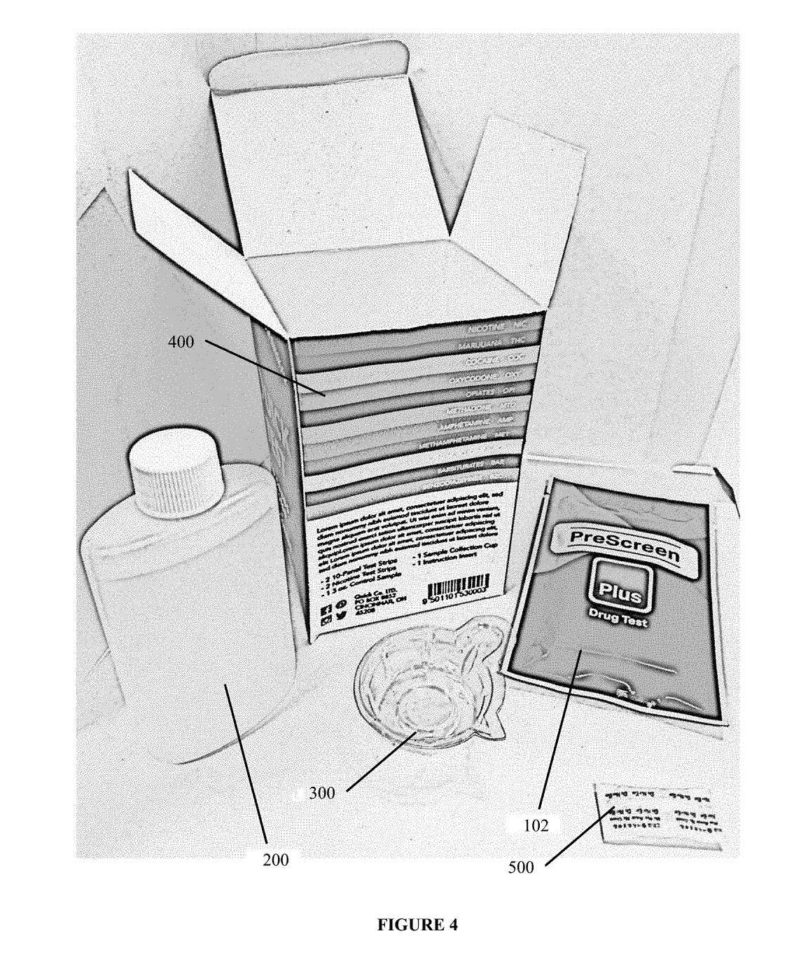

At least two multi-channel testing components 100 are provided in a moisture-resistant package 102, such as a hermetically sealed foil pouch as illustrated in FIG. 4. The package 102 may contain one or more silica gel packs, silica beads, and/or other common desiccants to prevent unwanted moisture ingress that could otherwise potentially contaminate or bias one or more of the testing strips contained in the device. Additionally or alternatively, desiccant packets may be provided in the storage box 400 as described below.

The storage box 400 itself may also be moisture resistant, with a single or mutely-lined metalized foil pouch providing such resistance. Ideally, the combination of package materials and desiccant(s) renders the packaging for the device virtually waterproof.

The testing component 100 itself includes a plurality of integrally formed test paper strips 110. A plastic cartridge body 130 is overmolded, formed, or assembled to receive and carry the strips 110 in a manner that leaves exposed portions 112. A protective cap 140 is snap-fitted to the body 120 to cover and protect portions 112 until the tester is ready for use. In lieu of exposed portions 112, it is also possible to provide a tube or parallel elongate members which rely upon capillary action to draw the test liquid into the cartridge 130 through appropriate apertures and tubes positioned immediately proximate to the strips 110 held in the body 120 of cartridge 130.

The strips 110 should be spaced apart to avoid unwanted contact, and each strip 110 may be made of differing indicator papers. The indicators are selected intended to reveal the presence of analytes at predetermined levels. In turn, the concentration of each indicator is sufficient to reveal the presence of analytes connected to the recent use of a targeted set of substances, such as nicotine, alcohol, marijuana (in the form of tetrahydrocannabinol and/or cannabinoids), steroids, cocaine, oxycodone, opiates, methadone, amphetamine, methamphetamine, phencyclidine (i.e., PCP), barbiturates, benzodiazepines, and the like. Use of other indicators for analytes associated with certain medical or physiological conditions may also be possible. Further still, with a multi-channel test component, it is possible to select complimentary or inter-related subsets of tests. For example, disparate drugs of abuse could be paired into a single test kit and/or physiological conditions, such as pregnancy, could be paired with unwanted or potentially dangerous substance uses, such as nicotine, alcohol, or drugs. Most usefully, indicators may be selected to test for any number and combination of analytes that may be indicative of recent cigarette, prescription drug, and/or controlled substance use.

The strips 110 run through the main body portion 120 of catridge assembly 130. The strips wick the test liquid (i.e., the urine specimen or the control) up into the portion that contains the analyte-detecting substances. A color change within the indicator or some other physical change occurs and is displayed through apertures 132 positioned to expose the relevant portion of strip 110 (as it is held within assembly 130). Comparative indicia 134 and/or a protective transparent cover (not shown) may be positioned on the outer surface of the assembly 130 proximate to the apertures 132 to facilitate in interpreting the test results.

In practice, the presence of a particular analyte (i.e., a positive test result) causes a marking to appear on only one reference portion of the strip, while the absence of the analyte causes two separate indicators to appear in the reference portion and in the test portion. Preferably, these indicators are simply lines or dots. A third result can be yielded to indicate an invalid test owing to contamination of the test strip or other problems. Such invalid tests return an indication only in the test portion but not in the reference portion.

A series of apertures 132 or viewing windows are provided in the cartridge. Each test strip 110 held in the cartridge 130 has its own viewing window/aperture, with an indicator on the cartridge to facilitate quick identification of the substance associated with the test results displayed in the window/aperture.

The test strips 110 and apertures 132 may aligned in an array so as to allow for the compact and efficient use of cartridge volume. In one embodiment, two rows of five separate test strips are provided, allowing for testing of up to 10 different substances. Alternatively, at least one pair of test strips 110 may test for the same analyte to allow for validation of a positive result. As noted above, specific test strips 110 and/or indicators can be paired to identify interrelated, significant, or correlated aspects of a test.

Of course, the return of a positive result, or even a pair of positive results, does not definitively establish use of the analyte in question. Indeed, many consumers would prefer an independent verification system similar to what would be used in professional laboratories. Especially in the event controlled substances are involved, it is impractical to package or otherwise provide for individual positive-result controls as verification means.

Therefore, the inventive system includes a multi-factor negative-result confirmed negative control liquid 200. This multi-factor control includes a substance that has a similar, if not identical, chemical composition to what would normally be detected in regular (i.e., unadulterated) human urine. However, shelf-life extenders, such as biocides, can be included to ensure long-term viability of the test kit. Examples, all of which are incorporated by reference and form certain aspects of the invention, are disclosed in U.S. Pat. Nos. 7,193,766; 9,128,105; and 9,316,648.

The liquid 300 may be contained within a capped dispensing bottle or other similar hermetically sealed containers. Sufficient quantity should be provided to allow for at least one user test (i.e., the volume of the test bin 300 plus a reasonable allowance for human error). Anywhere from 2 to 4 fluid ounces should be sufficient, with about 3 ounces (˜90 millimeters) being preferred.

Additionally, the mutl-factor negative control may include one or more surfactants which may facilitate the wicking/absorption process. These surfactants may foam when negative control fluid is transferred, although it will not negatively impact the results of the test kit. Colorants may also be added to make for easy identification of the negative control as compared to the actual sample. Common, non-reactive dyes range in colors including red, yellow, orange, green, and blue.

Notably, urine is chemically distinct from water, and local variations in water quality would make the use of normal tap water or even bottled water untrustworthy. By the same token, relying on a chemically similar control to that of unadulterated human urine will also account for a controlled verification test that tracks ambient, environmental conditions inherent to the original sample test. Thus, the provision of a verified negative result solution in the test kit is a key feature.

Further, to the extent human urine exits the body at an elevated temperature (usually between 32° to 36° C.), the kit may also include a warming device 500. This warming device may comprise a combustible, electric, or chemically-activated heat pack. Upon activation, the warming device is placed proximate to the multi-factor negative-result control to more closely mimic conditions inherent to the original specimen tested. In this manner, any variations in the test strips or overall urinanalysis conditions that might be influenced by the temperature of the sample are more closely accounted for and simulated, thereby further improving confidence in the results.

The kit also includes a test bin 300. Bin 300 is formed as a cup with a deep body well 310 that receives the test liquid (either via collection by the user or via a transfer vessel or sterilized cup). A spout 320 may be integrally formed within one or more of the sidewalls of the well 310 to facilitate the transfer and disposal of tested liquids, which can be discarded within an appropriate sanitary sewer.

In one embodiment, a handle 330 is provided to assist the user. In another embodiment, the ledge 340 is provided. Ledge 340 includes a retaining lip 342 to so that the test device 100 can rest on the ledge 340 while having the exposed portions 112 (or capillary tubes) extend sufficiently into the well 310 for immediate wicking and accurate test results.

A second multi-channel test device, also hermetically sealed, should be included in the test kit. This second device should be identical in every respect with the original device 100 intended for use with the actual urine sample. A second test or collection bin 300 can be provided to allow for side-by-side comparison.

In some embodiments, the container holding the control sample 200 may be formed to include a channel, slot, or flattened portion to accommodate and/or restrain the warming device 500. Additional shape may be provided (e.g., angled or sloping sides) to encourage convective heat transfer within the control/fluid held within the container. A removable cap, an integral syringe and/or pipette, a spout, and/or other means to facilitate extracting the sample and selectively disposing it within a desired location may also be formed in the container bottle.

In use, the user would only consult the negative-result control in the event a positive sample result is first returned. The user would then follow the same procedures as used for the sample test. In this manner, the user has an independent means of verifying the accuracy of the test kit and testing conditions without having to wait. Further, by enabling the user to conduct the tests instantaneously (the test cartridge returns results in under one minute under normal conditions), the possibility of loss or contamination of the specimen in route to a remote testing lab is eliminated. The procedure also ensures total privacy for the user, while sparing the potential embarrassment associated with monitored testing.

In use, urine is directly collected or poured into the well 310 of the first bin 300. If an intermediate collection cup is used, such device must be clean and sterile. Optionally, negative control 200 is dispensed into a second bin 300 (either at the same time as the sample or after the sample results were read). Care must be taken to avoid mixing or contaminating the contents of either bin 300 once the test liquids are in place.

The devices 100 are removed from their moisture-barrier packet 102, and cap 140 is detached from cartridge 130 to expose the strips 110 (but without making any contact with the strips 110 once they are exposed). When present, the devices 100 rest on ledge 340 or analogous structure of bin 300 or are held in place. The exposed portions 112 are fully submerged in the test liquid, but without touching the bottom or sides of well 310, for 10-15 seconds. The cartridges 130 are then removed and the caps 140 are snapped back on. The assembly is then left flat to allow for wicking for about 5 minutes.

In some embodiments, the indicators applied to strip 110 will provide two separate lines to indicate a negative test, whereas a single line is indicative of a positive test (if in the upper or “control” portion denoted by a C) or an invalid test (if in the lower or “test” portion denoted by a T). An invalid test may be indicative of insufficient specimen volume, contamination of the device 100, adulteration of the sample (by way of adding substances such as bleach), or possibly a faulty device 100.

Any variety of test strips can be employed. A combination of the most commonly used and/or abused substances would be particularly helpful, especially for parents and guardians of individuals who may be suspected of having an issue. In some embodiments, amphetamines, barbiturates, benzodiazepines, cocaine, tetrahydrocannabinol (marijuana), methamphetamine, opioid analgesics, phencyclidine, methylenedioxymethamphetamine (MDMA or ecstasy), morphine, oxycodone, phencyclidine, and nicotine.

The appropriate test window for analytes indicative of these substances must account for the time it takes for the body to produce the analytes after ingestion. For example (and as reflected in table 1 below), oxycodone analytes appear in urine in only 1-3 hours after ingestion, but these analytes can only be detected for period of 1-3 days. In contrast, MDMA appears within 2-7 hours after use but persists for 2-4 days. Consequently, the selection of substances for a given test kit must account for these varying windows, and it may be futile to include certain combinations. Information from Attachment 1 can, therefore, be used to identify appropriate combinations for multi-channel test kits.

The kit may also provide a separate, sterilized specimen collection cup (not shown). This cup should be made from an appropriate material, such as durable polymers or glass. Separately, a similarly sterilized control comparison cup is also provided. These cups may be of similar or identical construction, so as to further ensure the control testing proceeds under the same conditions as the original specimen. Also, the cups should be configured to cooperate with the test cartridge without any of the plurality of test strips carried in the cartridge from coming into contact with the sides of the cup (as such contact could produce inaccurate or invalid results).

As with the test cartridges, the sterilized cups and/or bins 300 should be packaged in a manner to preserve their integrity. However, moisture is not as significant of a concern, so there may not be a need to include the cups in the foil/moisture-proof packaging.

TABLE 1
Exemplary Analyte Characteristics

| | Minimum time | | |
|---|---|---|---|
| | for analytes | Maximum time | |
| | to become | for detection | |
| Substance | present in urine | of analytes | |
| Amphetamine (AMP) | 4-6 hours | 2-3 | days |
| Bariturates (BAR) | 2-4 hours | 3-15 | days |
| Benzodiazepines (BZO) | 2-7 hours | 5-10 | days |
| Cocaine (COC) | 2-6 hours | 2-3 | days |
| Marijuana (THC) | 1-4 hours | Up to 30 days | |
| Methamphetamine (MET) | 4-6 hours | 2-3 | days |
| Ecstasy (MDMA) | 2-7 hours | 2-4 | days |
| Morphine (MOP300) | 2-6 hours | 1-3 | days |
| Oxycodone (OXY) | 1-3 hours | 1-3 | days |
| Phencyclidine (PCP) | 4-6 hours | 7-14 | days |

As previously noted, individuals may have a range of reasons for seeking verified, in-home testing. However, a positive result does not necessarily indicate impairment or intoxication. Instead, this test kit is envisioned as a means of identifying and/or intervening in instances of substance abuse or other dangerous or life-threatening behaviors. However, certain foods, medicines (over the counter or prescription), and dietary supplements may provide false positive results even when the negative control is employed. However, the advantage of the negative control is that it provides an independent datapoint, thereby reducing the tested subject's ability to allege a problem in the testing procedure. Further, by providing instantaneous access to negative-control-verified result, it eliminates the scenario where the test subject submits a second sample and disappears before any preliminary verification can be rendered.

In view of the foregoing, a method for conducting urinalysis is contemplated. This method provides the advantages of privacy and enhanced confidence in the results as noted above. At its core, the method includes providing a test kit including a plurality of identical multi-channel test devices and a negative control sample. Optionally, a warming pack and/or one or more sterilized sample bins may be included. All components have the characteristics noted above.

The individual collects a urine sample and partially submerges one test device therein for a predetermined period of time. Separately, a second test device is similarly, partially submerged in the negative control sample. A comparison of the results produced by each test device is made to identify commonalities or areas for potential mistakes. For example, if the same channel indicates as “invalid” for the test urine and the negative control, the user is alerted to the likelihood of a problem with the procedure or with the test devices themselves. Conversely, if urine sample returns positive or invalid while the negative control indicates negative, the user has an increased likelihood that the test is positive or that the urine sample was improperly adulterated or handled. In all instances, the feedback provided to the user is instantaneous, and appropriate follow up actions can be taken without the need to wait for a second round of testing.

Another aspect of the invention is a preparation of individualized urinalysis kits. This method involves identifying a subset of characteristics the individual (or a guardian or other caretaker for the individual) wishes to monitor. A specific combination of test strips including indicators for analytes associated with the identified characteristics are then encased within at least two separate multi-channel test device. Each multi-channel device is separately, hermetically sealed—with an optional desiccant packet provided within a foil pouch that holds each device. At least two of the sealed device/optional desiccants are then coupled with a container including a negative control and a corresponding number (i.e., equal to the number of devices) of test bins for holding samples to create an individualized test kit. The test kit is then boxed, optionally including a warming packet, and shipped to the individual.

A still further aspect of the invention involves providing a negative control to improve confidence in results of an in-home urinalysis. The negative control delivers an enhanced level of confidence for concurrently performed home urinalysis using a multi-channel device. The analytes tested by the multi-channel device are selected based upon the needs of the tested individual. The negative control is formulated to include compounds commonly found in urine samples, although an optional surfactant and/or colorants could be added to facilitate use of the negative control in the in-home urinalysis method. In substance, the method involves providing a first urinalysis test device for the detecting analytes in a urine sample that are indicative of recent use or ingestion by an individual of selected substances. Upon indication of a positive test for such analytes or concurrent with use of the test device, a negative control is provided along with a second urinalysis test device that is substantially identical to the first device. Said negative control is tested by the second test device to improve confidence in results provided by the first test device. The selected substances may be identified according to circumstances unique to the individual.

Finally, the negative control kit, as well as the above referenced methods, may also be particularly helpful in the realm of pregnancy tests. Here, the “analyte” of interest is actually a hormone passed in the urine, human chorionic gonadotropin (hCG). Test strips to test for the presence of hCG are well known, and they may be incorporated into the multi-channel device. When coupled with a panel for certain drugs and/or alcohol, the resulting multi-channel device may be of particular utility, in terms of identifying unwanted and dangerous use of certain substances by women who are pregnant. The remaining principles of the test device are similar to those identified above, although some indicators may require greater resident time within the sample.

In this context, the need for instant, accurate, and verified results becomes even more definitive. By coupling a negative control, it provides a level of confidence as to whether the individual performing the test incorrectly performed any of the procedures associated with.

Other physiological conditions involving the passing of hormones or related substances can be substituted or added to the kit and methods. That is, while the term “analyte” was initially provided within the context of compounds produced by the body as a result of metabolic reactions on a selected substance (i.e., certain drugs or foods), test strips and indicators that detect human (or artificial) hormones and other substances produced by the body, but only under specific conditions and/or as might be indicative of certain conditions, can be easily incorporated into the kit and methods. As with pregnancy, this affords the kit designer and the individual with greater flexibility to create individualized and/or more meaningful and verifiable test results—all without having to rely upon the intervention of a test lab, the long lead times, the transfer and contamination risks, and the other drawbacks mentioned above.

Testing for the characteristics of the urine sample itself may be also be useful, in terms of providing additional information and feedback in a simple, in-home test kit. Conditions or information about a urine sample that can be easily determined with a test strip include: the presence of glucose, ketones, or nitrites, the pH or specific gravity of the sample itself (with the latter being particularly important to ensure the sample does not get flagged as being diluted/adulterated), and the presence of blood (by way of hemoglobin or myglobin). Additionally, test strips for urobilinogen, which is indicative of liver damage, could be particularly useful when coupled with a panel of strips for common substances of abuse, while a test strip for vitamin C may be included so as to provide further insights on potential adulteration of the urine sample. Ultimately, the nature and implications of these individual characteristics are well documented in contemporaneous literature, although providing these insights within the context of an instantaneous test that provides side-by-side results as to potential substance abuse and/or other conditions is heretofore unknown to the inventor, at least in the in-home consumer market. As above, all these items are coupled with a contemporaneous negative control to provide the tester with an added level of confidence to the extent any one or combination of items comes back positive.

In view of the array of possibilities, the customization of test kits becomes even more significant. For example, a pregnancy kit coupled with certain alcohol and drug tests allows for instantaneous identification of risky behaviors. Similarly, for guardians, parents, or concerned individuals, the ability to include test strips to identify adulterated urine samples, as well as tests for the substances of concern themselves (e.g., opioids, marijuana, etc.), may be particularly useful. In all instances, the use of an independently provided negative control provides a level of real-time confidence in the test results that was not necessarily available when only a single urine sample is tested.

For the sake of completeness, any and all of the foregoing methods are informed by the details of the home test kit described above. Similarly, the references incorporated by reference inform certain aspects of the components of the test kit. Finally and most importantly, the ability to select and ascertain a range of disparate but related and useful information in a single test kit is another unique aspect of the invention.

Although the present embodiments have been illustrated in the accompanying drawings and described in the foregoing detailed description, it is to be understood that the invention is not to be limited to just the embodiments disclosed, and numerous rearrangements, modifications and substitutions are also contemplated. The exemplary embodiment has been described with reference to the preferred embodiments, but further modifications and alterations encompass the preceding detailed description. These modifications and alterations also fall within the scope of the appended claims or the equivalents thereof.

What is claimed is:

1. A test kit for verifying urinalysis results with improved levels of confidence, the kit comprising:
a number of identical test devices each holding a plurality of urinalysis test strips arranged in a spaced apart array so that wicking or capillary action draws a test sample into contact with a discrete indicator disposed on or in each strip;
a negative-result control sample including multi-factors selected to mimic human urine; and
a plurality of sterilized test bins, matching the number of test devices, and each test bin has a sufficient depth to submerge the array of urinalysis test strips without having any single test strip come into contact with a sidewall of that bin; and
wherein each test device is contained within a separate protective covering prior to use and wherein the number of test devices is at least two.

2. The test kit of claim 1 wherein the indicators are selected to be associated with any combination of two or more of the following: use of a drug within a window of at least 2 hours to 2 weeks prior to exposure of the indicator to the test sample; pregnancy; presence of glucose, ketones, nitrites, vitamin C, urobilinogen, and/or blood; pH of the test sample; and specific gravity of the test sample.

3. The test kit of claim 2 wherein the drug is at least one selected from: nicotine, tetrahydrocannabinol, cocaine, amphetamine, methamphetamine, opioid analgesics, phencyclidine, methylenedioxymethamphetamine (MDMA), barbiturate, and benzodiazepines;

4. The test kit of claim 1 wherein the discrete indicator on each test strip is concealed within a cartridge holder of the test device proximate to a viewing portal for said test strip.

5. The test kit of claim 4 wherein written indicia is provided on the cartridge holder immediately proximate to at least one of the viewing portals, said indicia facilitating identification of a test result.

6. The test kit of claim 1 wherein each test device is contained in a separate foil pouch.

7. The test kit of claim 6 wherein a desiccant is provided within each pouch.

8. The test kit of claim 1 wherein each test device is provided with an overcap that is selectively detached from the test device to expose a pathway for contact between the test sample and the indicators.

9. The test kit of claim 1 wherein the multi-factor negative-result control sample includes at least one of shelf-life extenders, a surfactant, and a colorant.

10. The test kit of claim 1 contained within a hermetically sealed package.

11. A method of verifying urinalysis results, the method comprising:
providing the test kit of claim 1;
collecting a urine sample from a tested individual;
depositing the urine sample in a first test bin;
providing the multi-factor negative-result control to a second test bin;
contacting a first test device with the first bin to submerge a distal portion of the first device without contacting any part of the first test bin, thereby exposing the urine sample to the indicators of the first test device for a selected period of time;
contacting a second test device with the second bin to submerge a distal portion of the second device without contacting any part of the second test bin, thereby exposing the multi-factor negative result control to the indicators of the second test device for the selected period of time; and
comparing characteristics of the indicators of the first and second test devices to verify positive and/or negative results produced by the first test device.

12. The method of claim 11 wherein the indicators are selected to reveal at least one of: use of a drug within a window of at least 2 hours to 2 weeks prior to exposure of the indicator to the urine sample; pregnancy; presence of glucose, ketones, nitrites, vitamin C, urobilinogen, and/or blood; pH of the urine sample; and specific gravity of the urine sample.

13. The method of claim 12, wherein the indicators selected reveal use of a plurality of different drugs and pregnancy.

14. The method of claim 12, wherein the indicators selected reveal use of a plurality of different drugs and at least one selected from: presence of glucose, ketones, nitrites, vitamin C, urobilinogen, and/or blood; pH of the urine sample; and specific gravity of the urine sample.

15. The method of claim 14 wherein the contacting the first device with the urine sample occurs simultaneously with the contacting the second device with the multi-factor negative-result control to ensure the selected period of time is identical for each.

16. The method of claim 11 further comprising warming the multi-factor negative-result control prior to contact with the second device.
